(19) United States
(12) Patent Application Publication
(10) Pub. No.: US 2014/0142294 A1
(43) Pub. Date: May 22, 2014

(54) METHOD FOR MANUFACTURE OF DEXTRAN, DEXTRAN SOLUTION OBTAINED, AND USES

(71) Applicant: Marco Wieser, Saint Etienne (FR)

(72) Inventor: Marco Wieser, Saint Etienne (FR)

(73) Assignee: S.P.C.M. SA, Andrezieux Boutheon (FR)

(21) Appl. No.: 14/127,740

(22) PCT Filed: Jun. 18, 2012
(86) PCT No.: PCT/EP2012/061619

§ 371 Date: Dec. 19, 2013

(30) Foreign Application Priority Data
Aug. 1, 2011 (FR) 1157048

(51) Int. Cl. C12P 19/08; C08B 37/02

(52) U.S. Cl. CPC C12P 19/08; C08B 37/0021
USPC 536/112; 435/103

(57) ABSTRACT

Microbiological method of production of a dextran solution, according to which a culture medium containing sucrose is inoculated with a preculture of a bacterial strain that is able to produce dextran, then the dextran solution obtained at the end of fermentation is recovered directly, without a subsequent concentration step, characterized in that:—before inoculation, the culture medium contains at least 10 wt. % of sucrose,—after inoculation, sucrose is added again in conditions such that the total amount of sucrose in the medium, including that present before inoculation, is at least 16 wt. %,—the dextran solution obtained contains at least 10 wt. % of dextran. The native solution of dextran obtained and use of the solution as a flocculant.

### TECHNICAL FIELD OF THE INVENTION

The present invention relates to the field of polysaccharides obtained microbiologically, more particularly dextran. It relates more precisely to the manner of production of the latter as well as its use as a flocculant of aqueous suspensions of solid particles and as a thickener of aqueous media.

Dextran is a long, complex molecule resulting from the transformation of sucrose by the action of enzymes called dextransucrases, which are secreted by bacteria such as for example Leuconostoc mesenteroides. Dextran can thus be produced from a bacterial culture of the aforementioned type in a specific culture medium.

### Prior Art

It is known that certain microorganisms can be cultivated in such a way that they produce extracellular enzymes capable of transforming sugars to microbial polysaccharides. Said polysaccharides include for example xanthan, scleroglucan, schizophyllan, diutan, levan, gellan, welan, pullulan and dextran.

Dextrans are notably produced from a bacterium such as for example Leuconostoc mesenteroides cultivated in a sucrose-supplemented culture medium. Dextran is obtained in practice from the polymerization of the glucose monomers of sucrose to glucan (polyglucose) by the dextransucrases of the bacterium. The culture broth generally contains, non-exhaustively, besides sucrose, nutrient elements such as sources of nitrogen, for example ammonium salts, urea, hydrolysates of milk and meat proteins, for example casein, a yeast extract, as well as buffer salts such as potassium or sodium phosphates, minerals such as iron, magnesium, calcium, and manganese, vitamins and an anti-foaming agent. As already noted, this broth constitutes the fermentation medium in which the bacteria will produce the enzymes and in which the sucrose will be transformed to dextran.

For example, document Karthikeyan et al. (Optimization of batch fermentation conditions for dextran production,) discloses a process for dextran production from a bacterial culture of Leuconostoc mesenteroides fermentation of sucrose. The concentration of dextran in the final solution is about 13.5 wt %.

Document SK 286 070 discloses a similar process, yielding a solution containing about 5.3 wt % of dextran.

Document DD 226 015 discloses a similar process, with a final concentration of dextran about 6.2 to 6.3 wt %.

The production of dextrans in bacterial culture is normally followed by at least one step of purification of the dextran obtained after fermentation. Purification is usually carried out by precipitation with alcohol, such as ethanol or isopropanol. Cells, hydrolysing enzymes that may degrade the polymer, as well as the medium, can be removed from the dextran in this purification. It thus makes it possible to obtain a dextran that has better performance, in particular for application as a flocculant in mines.

Thus, the article Production of Dextran by newly isolated strains of Leuconostoc mesenteroides PCSIR-4 and PCSIR-9 (Turkish Journal of Biochemistry—2005; 31 (1); 21-26) describes a method of production of dextran employing steps of precipitation and purification. The purification step described comprises several substeps of precipitation, centrifugation or filtration of the precipitate, washing, drying and dissolution, making the manufacturing process complex, long and expensive.

The article also teaches that the optimum total sucrose concentration is 10%, and it is stipulated that the sucrose is added just once. When the sucrose concentration is higher, inhibition of dextran synthesis is observed. The dextran concentration obtained in this article is at most about 9%, for an amount of sucrose employed of 30%. In general, the dextran concentrations, after fermentation, obtained microbiologically are of the order of 1 to 7 wt. %, most often between 2 and 5 wt. % [Behravan et al. 2003 Biotechnol Appl Biochem 38:267-269]. Now, the mining industry, including the alumina mining industry, usually employs dextran solutions at 15 wt. %. This therefore requires at least one step of concentration of the dextran solution.

The molecular weight of the dextran produced is also important. Dextran is widely used in the medical field as transport media in the blood. In this case it has a low molecular weight, between 1000 and 70 000 g/mol. It is also used in the areas of enhanced oil recovery, cosmetics and foodstuffs, where it serves as a thickener. It is also employed in papermaking, drilling and mining, for example in alumina mines, where it serves as a flocculant (for example, see document U.S. Pat. No. 3,085,853). For good efficiency as a flocculant, usually a high molecular weight is required, generally above 100 000 g/mol.

### The Technical Problem to Be Solved

It therefore appears that the microbiological production of dextran is limited, because without a concentration step it cannot produce solutions with dextran concentration, after fermentation, above 10%. Moreover, it has been found to be necessary to purify the dextran produced as a result of fermentation, to obtain good performance in flocculation or thickening. Of course, the purification steps tend to increase the cost of the finished product.

In other words, the problem to be solved by the invention is to develop a method of microbiological production of dextran that makes it possible to increase the concentration produced, without a subsequent concentration step. Another objective is to develop a method of production of dextran that does not require a subsequent purification step, the dextran obtained being just as effective as purified dextran, especially when it is used as a flocculating or thickening agent.

### DESCRIPTION OF THE INVENTION

The present invention aims to overcome the aforementioned problems.

Thus, the applicant has developed a new solution of dextran obtained microbiologically at high concentration, i.e. whose dextran concentration after fermentation is, without a concentration step, above 10 wt. %, namely by optimizing the polymerization parameters during fermentation.

In particular, the applicant found that this objective could be achieved by incorporating a certain amount of sucrose in the culture medium, prior to inoculation of the bacterium, and adding sucrose after inoculation.

More precisely, the invention relates to a microbiological method of production of a dextran solution, according to which a culture medium containing sucrose is inoculated with a preculture of a bacterial strain able to produce dextran, then the dextran solution obtained at the end of fermentation is recovered directly, without a subsequent concentration step.

The method is characterized in that:
before inoculation, the culture medium contains at least 10 wt. % of sucrose,
after inoculation, sucrose is added again in conditions such that the total amount of sucrose added to the medium, including that present before inoculation, is at least 16 wt. %,
the dextran solution contains at least 10 wt. % of dextran.

Thus, the applicant found that adding sucrose at least twice made it possible to avoid the phenomenon of inhibition of bacterial growth and of the activity of the enzyme dextransucrase.

Moreover, after inoculation, sucrose is added again in conditions such that the total amount of sucrose, including that present before inoculation, is at least 16 wt. %.

According to the invention, addition of sucrose after inoculation can be continuous or discontinuous.

When addition of sucrose after inoculation is carried out several times, it is advantageously at least twice.

Addition at least twice is necessarily a separate addition. Nevertheless, each addition can be carried out instantaneously or continuously until the desired amount has been introduced. It is also possible to combine these two possibilities.

In other words, each addition is carried out instantaneously or continuously or the first addition is carried out continuously and the second addition is carried out instantaneously or vice versa until the desired amount has been introduced.

For further improvement of the final concentration of the dextran solution, the total amount of sucrose added, including that present in the supplemented medium, is at least 20 wt. %, preferably at least 25 wt. %.

The dextran solution preferably contains at least 14 wt. % of dextran, and more preferably at least 18 wt. % of dextran.

According to the invention, the strain that is able to produce dextran is a strain of the genus Leuconostoc, Streptococcus, Lactobacillus or Acetobacter. In a preferred embodiment, it is a strain of Leuconostoc mesenteroides, in particular the strains NRRL B 1299, NRRL B 742, NRRL B-512 F, NRRL B 523, V-2317 D, KCTC 3505, ZDRAVLJE SR.P.

Accordingly, the method of the invention makes it possible to obtain, without a concentration step, i.e. directly at the end of fermentation, a dextran solution with a concentration above 10 wt. %, very preferably above 14 wt. % and even more preferably above 18 wt. %.

The dextran solution obtained substantially need not be purified, including up to the time of use. To put it another way, in this case the cellular materials and/or fermentation medium are not separated from the dextran solution. In other words, it can contain some or all of the cellular materials and fermentation medium. In particular, the solution can contain bacteria, enzymes, as well as salts and elements of the culture broth.

To promote growth of the microorganisms, the level of oxygenation of the culture medium is increased. In practice, oxygenation is of the order of 0.25 vvm at the start of fermentation, a step during which multiplication of the microorganisms is desired. Secondly, oxygenation is decreased to promote the formation of the enzyme and its activity and thus promote the biopolymerization of sucrose to dextran. In practice, oxygenation is decreased for example to a value of the order of 0.1 vvm.

The unit vvm corresponds to the volume of air introduced into the medium relative to the filled volume of the reactor in which the production of dextran takes place.

As will be seen later, the dextran produced has a high molecular weight. Accordingly, the fermentation medium is fairly viscous. The stirring of the fermentation medium must be sufficient to ensure mass transfer between the enzyme dextransucrase and the sucrose and thus promote homogeneity of fermentation throughout the fermenter. In practice, the fermentation medium is stirred at between 100 and 1000 rev/min, preferably between 200 and 500 rev/min. A person skilled in the art is perfectly capable of adjusting this parameter in relation to the growth of the microorganisms, the oxygen concentration and the sucrose consumption created by stirring.

It is known from the prior art that fermentation transforms a proportion of the sucrose to acid, which causes a drop in pH, generally from 7.2 to 4.3. Three phases are observed:
the first, during which the pH is adjusted to 7.2, the optimum condition for growth of the microorganisms;
the second, during which the pH is adjusted to 6.6, the optimum condition for formation of the enzymes;
the third, during which the pH is adjusted to 5.2, the optimum condition for the formation of dextran.

To optimize these 3 phases, the pH is adjusted conventionally by controlled continuous addition of sodium hydroxide or of ammonia in order to control the pH to a constant value of 7.2, or 6.6 or 5.2. This operation requires expensive special equipment.

The applicant found that, surprisingly, the pH could be controlled by adding an amount of buffer much greater than that usually encountered, without limitation and without additional equipment.

More precisely, the initial pH is increased to a value above 7.5, preferably above 8. Then, according to the invention, the drop in pH is controlled by means of a large amount of one or more buffer salts. In particular, during fermentation, whereas an amount of 0.1 wt. % of buffer salt, for example disodium or dipotassium phosphate, is usually employed, an amount of at least 0.5 wt. % of at least one buffer salt, advantageously at least 1 wt. %, preferably at least 2 wt. %, is added according to the invention. This makes it possible to control the drop in pH and optimize the durations of the phases of growth of the microorganisms (optimum pH at 7.2) and formation of the enzyme (optimum pH at 6.6), and formation of dextran (optimum pH at 5.2) without the limitation of programming and regulating three different pH values by adding a solution of base of the sodium hydroxide or ammonia type.

Other buffer salts can also be used, for example ammonium phosphate, sodium borate or sodium citrate.

Despite optimum growth at pH 7.2, it is found, as already mentioned, that a satisfactory growth rate is obtained with a starting pH at pH 8.0. In this preferred embodiment, fermentation begins at pH 8.0. It allows accumulation of dextran from the very start of fermentation, thus obtaining a final dextran concentration above 10 wt. %, preferably above 14 wt. % and more preferably above 18 wt. %.

Advantageously, fermentation is stopped at a pH above 5, preferably 5.4, optionally 5.3, which is above the conventional values of 4.5-4.8. The applicant found, surprisingly, that stopping fermentation at pH 5.4, optionally 5.3, made it possible to improve the flocculation and thickening performance of the dextran solutions thus produced. Moreover, the dextran formed and obtained with a stopping pH at pH 5.4, optionally 5.3, corresponds to a maximum viscosity of the dextran.

The temperature at which culture and biotransformation take place is in practice between 15 and 35° C. In a preferred embodiment, the initial temperature is between 25 and 35° C. for a period of time between 4 and 8 h, which makes it possible to optimize the growth of the microorganisms. Then the temperature is lowered and is between 18 and 24° C. for the rest of the fermentation, i.e. for a period between 10 and 30 hours, for optimizing the formation of the enzymes and of dextran.

In a preferred embodiment, and in contrast to the usual techniques, fermentation, comprising the steps of production of the bacterial strain, production of the enzyme and production of dextran, is carried out in the same reactor, which simplifies production.

Quite surprisingly, and in contrast to regular practice, the method can be carried out in a substantially unsterilized reactor.

Advantageously:
the water is prefiltered before use, with filters with diameters below 1 μm, preferably less than or equal to 0.3 μm;
the air is prefiltered before use, with filters with diameters below 0.5 μm, preferably less than or equal to 0.3 μm;
the inoculum ratio is between 0.5 and 30, preferably between 1 and 20, very preferably between 5 and 10. The inoculum ratio corresponds to the volume percentage of the preculture solution added to the fermenter relative to the total volume in the fermenter.

According to the invention, the molecular weight of the dextran of the invention varies between 100 000 and 500 million g/mol. Preferably it is above 500 000 g/mol and very preferably between 1 and 50 million g/mol.

The invention thus also relates to a native solution of substantially unpurified dextran containing at least 10 wt. %, advantageously at least 14 wt. %, preferably at least 18 wt. % of dextran, with a molecular weight between 100 000 and 500 million g/mol that can be obtained by the method described above.

The expression “native solution of dextran” denotes a dextran solution obtained at the end of fermentation of a bacterial culture of the kind stated above supplemented with sucrose, the solution being neither substantially purified, nor concentrated at the end of fermentation. The dextran solution is therefore used as it is, in the presence of the impurities present in the solution. The impurities are, in particular, bacterial cells, components of lysed cells including lipids, proteins and nucleic acids, enzymes, the salts of the medium, unconverted sucrose, other sugars such as monosaccharides, for example fructose, di- and oligosaccharides, buffer salts, oligopeptides, organic acids such as lactic acid or amino acids, as well as other ingredients of the medium.

According to the invention, the native dextran solution contains at least 2 wt. %, preferably at least 5 wt. %, advantageously at least 8 wt. % of impurities, notably some or all of the impurities stated above. The concentration of impurities is measured by subtracting the dextran concentration from the total concentration of dry matter of the native solution.

The dextran concentration is measured by gravimetry after precipitation of the native solution with 3 volumes of methanol and drying of the precipitate overnight at 105° C. The total dry matter is measured by gravimetry after drying the native dextran solution overnight at 105° C.

The invention also relates to the use of the native dextran solution defined above either as a flocculant, coagulant, precipitation agent, sedimentation agent or decanting agent, of aqueous suspensions of solid particles, or as a thickener of aqueous media.

More precisely, the dextran solution according to the invention can be used in many processes in which it is necessary to separate water from solid particles, which are of biological, organic or mineral origin. We may mention, for example, processes for treatment of municipal and industrial wastewater, processes for flocculation, coagulation and precipitation of particles and rheological modification and anti-dusting operations employed for mining effluents. This is notably the case for the flocculation of hydrated alumina resulting from alumina extraction by the Bayer process, or for flocculation of the effluents resulting from the exploitation of bituminous sands. The dextran solution can also be used as a growth modifier of mineral crystals.

The Bayer process is used for extracting aluminium oxide from bauxite by dissolving the bauxite in an extremely alkaline aqueous solution. At the end of dissolution, a mud, or digest, is obtained, which consists of water and mineral particles including hydrated alumina.

As already mentioned, the native dextran solution according to the invention can also be used as a thickener of aqueous media and notably in the areas of enhanced oil recovery and cosmetics.

The concentrations of dextran used for these applications can vary depending on the nature of the suspensions and the molecular weight of the dextran. In general, said amount varies between 1 g per tonne of total solids to 1000 g per tonne. Preferably the amounts used vary between 5 and 500 g per tonne of total solids.

For these applications, the molecular weights of the dextrans are relatively high. Dextrans according to the invention having a molecular weight above 100 000 g/mol give good results. However, those with a molecular weight of at least 500 000 g/mol, preferably between 1 and 50 million g/mol are preferred. In practice, the molecular weight will not exceed 500 million g/mol.

It is also quite unexpected to obtain excellent results in flocculation of aqueous suspensions of solid particles with a substantially unpurified dextran. In fact, it is generally assumed that the impurities of the culture medium and organic materials such as bacteria, enzymes, salts and organic components of the fermentation medium are undesirable for this type of application as they degrade flocculation performance. But surprisingly, the dextran solution of the invention is effective even if it is substantially unpurified.

In this type of application, the dextran solution can be combined with any other additive used conventionally, for example water-soluble polymers, and notably those based on acrylamide, acrylate, hydroxamate, polyamine, or diallyldimethylammonium chloride.

The invention and the advantages resulting therefrom will become clear from the following examples.

In the examples and in the description, the terms used have the following definitions.

The term inoculate means to transfer an amount of aqueous suspension of microorganisms into a larger amount of aqueous medium to permit development and multiplication of these microorganisms into a larger amount.

The term agar denotes a chemical compound generally used for gelation of a liquid medium in a Petri dish and that serves as substrate for development of the microorganisms.

The term MRS medium corresponds to a culture medium used conventionally whose composition is as follows per litre of aqueous preparation:
peptone 10.0 g
meat extract 8.0 g
yeast extract 4.0 g
glucose 20.0 g
sodium acetate trihydrate 5.0 g
ammonium citrate 2.0 g
Tween 80 1 ml
potassium hydrogen phosphate 2.0 g
magnesium sulphate heptahydrate 0.2 g
manganese sulphate tetrahydrate 0.05 g

### EXAMPLE 1

### Production of Dextran Solution at High Concentration Without a Concentration Step and Without Purification

A fresh culture of Leuconostoc mesenteroides B512 of less than one week was inoculated on MRS medium, in which the 20 g of glucose had been replaced with 20 g of sucrose, supplemented with 20 g/L of agar, 100 ml of the same medium without agar, previously sterilized for 20 min at 121° C. This culture is incubated for 24 h at 30° C., with stirring at 150 rpm. Then this preculture is transferred to 5 L of the same sterilized medium without agar. It is incubated in the same way and then transferred to 100 L of the same sterilized medium without agar. The same operation is carried out in 2000 L sterilized. This last preculture is inoculated in 14000 L of water, which contains the same medium without agar, supplemented with 16 wt. % of sucrose, 2.25% of dipotassium phosphate and 0.05% of Rhodorsil 481 anti-foaming agent. The medium is not sterilized but the water was filtered beforehand at 0.22 μm. After 5 h of fermentation at 30° C. and continuous addition of 0.25 vvm of air filtered at 0.22 μm, the temperature is lowered to 22° C. After 10 h of fermentation, it is lowered to 0.1 vvm of air and 6% of sucrose is added, and after 14 h, 6% of sucrose is added again. After 15 h of fermentation, the aeration is stopped. After 18 h, 18.6% of dextran dry matter is obtained in the fermentation broth, which contains biomass and other impurities.

### EXAMPLE 2

### Purification of the Dextran

50 kg of the native dextran solution obtained from the fermentation according to example 1 is diluted and mixed with 200 kg of deionized water. This mixture is filtered by dialysis, to remove all the salts from the medium and other impurities, on an ultrafiltration membrane of the hollow polysulphone fibre type with a cut-off of 500 kD tangentially. The salts of the fermentation medium that pass through the membrane are contained in the filtrate (that which passes through the filter). The dextran is concentrated by recirculation in the retentate (that which does not pass through the filter) up to the initial volume of the dextran.

50 kg of the retentate is diluted a second time with 200 kg of deionized water, and concentrated again by diafiltration and recirculation until 44 kg of dextran purified to a concentration of 14.7% is obtained at a yield of 69.2%.

### EXAMPLE 3

### Efficacy of Unpurified Dextran Relative to Purified Dextran

A reference purified dextran and those from examples 1 and 2 are diluted in 500 ml of deionized water to a concentration of 0.1% of active substance. They are stirred vigorously for 30 min on a stirring plate using a magnetic bar.

In parallel, to remove the variations in concentrations of hydrated alumina in the digests obtained by the Bayer process, the following operation is carried out. 80 g of dry hydrated alumina obtained from the alumina mine is mixed in 1 litre of Bayer liquor, or digest, taken directly from the alumina mining plant, then filtered to remove the hydrated alumina.

The tests are carried out using 1000-ml graduated cylinders placed in a bath heated to 65° C. The dextran is added at a rate from 2 to 20 g/t active substance relative to the amount of hydrated alumina. This unit is widely used in industry as the concentrations of hydrated alumina vary from one mine to another, and at one and the same mine from one day to the next. It is therefore necessary to adjust the amount of dextran in relation to said amount of hydrated alumina.

The sedimentation rate is measured in m/s in the range from 1000 ml to 700 ml. This is the time taken for the level of separation between the supernatant, which is clear, and the concentrated suspension to pass from the 1000 ml mark to 700 ml.

The clarity of the supernatant is evaluated by determining the amount of dry matter of the supernatant, obtained after 15 min of sedimentation, by taking 100 ml of supernatant from the graduated cylinder, and then filtering, drying (2 hours at 105° C.) and weighing the supernatant.

The compaction is evaluated by observing the deposit at the bottom of the cylinder and is measured after 10 minutes by means of the graduated cylinder. All the tests are performed three times and the mean value is found.

### Results

A. Alumina Mine in Lushan, China

| | Sedimentation | Dry matter | |
|---|---|---|---|
| | rate | of supernatant | Compaction |
| Flocculant (dosage 7 g/t) | (m/h) | (g/l, 15 min) | (ml, 10 min) |
| Purified dextran, Example 2 | 1.4 | 0.90 | 375 |
| Crude dextran, Example 1 | 1.3 | 1.35 | 390 |
| Reference (purified) 85701* | 1.3 | 1.45 | 410 |

The products 85700 and 85701 are purified dextran solutions, known as references in the hydrated alumina flocculation market. The applicant calculated the dextran concentration, which is around 15 wt. %, and the amount of impurities, which is 0.7 wt. %.

Alumina Mine at Paranam, Surinam

| | Dosage | Dry matter of supernatant |
|---|---|---|
| Dextran | (g/t) | (g/L, 10 min) |
| Blank | 0 | 2.24 |
| Example 1, crude | 5 | 0.35 |
| | 10 | 0.28 |
| Reference (purified) 85701 | 5 | 0.37 |
| | 10 | 0.28 |

B. Alumina Mine of St. Ciprian, Spain

| Dextran (2 g/t) | Dry matter of supernatant (g/L, 5 min) |
|---|---|
| Blank (without dextran) | 2.56 |
| Reference (purified) 85700 | 0.54 |
| Purified (example 2) | 0.51 |
| Crude (example 1) | 0.68 |

C. Alumina Mine at Zibo, China

| Dextran | Sedimentation rate | Dry matter of | Compaction |
|---|---|---|---|
| 10 g/t | (m/s) | supernatant (g/L, 5 min) | (mL, 5 min) |
| Blank | 1.5 | 1.45 | 240 |
| Reference | 2.4 | 0.38 | 200 |
| (purified) | | | |
| 85701 | | | |
| Crude | 2.7 | 0.36 | 195 |
| (example 1) | | | |

These tests are able to demonstrate the effectiveness of the new dextran as a flocculant of hydrated alumina even if it has not been purified. The unpurified dextran is equivalent to or surpasses the purified products 85700 and 85701, the reference products on the hydrated alumina flocculation market.

1. Microbiological method of production of a dextran solution, according to which a culture medium containing sucrose is inoculated with a preculture of a bacterial strain able to produce dextran, then the dextran solution obtained at the end of fermentation is recovered directly, without a subsequent concentration step, characterized in that:
before inoculation, the culture medium contains at least 10 wt. % of sucrose,
after inoculation, sucrose is added again in conditions such that the total amount of sucrose in the medium, including that present before inoculation, is at least 16 wt. %.

2. Method according to claim 1, characterized in that after inoculation, sucrose is added to the medium continuously or discontinuously.

3. Method according to claim 1, characterized in that sucrose is added after inoculation at least twice.

4. Method according to claim 1, characterized in that the total amount of sucrose added, including that present in the supplemented medium, is at least 20 wt. %, preferably at least 25 wt. %.

5. Method according to claim 1, characterized in that the pH is fixed at the start of fermentation at a value of 8, then the reaction is stopped at a value above 5.

6. Method according to claim 1, characterized in that during fermentation, at least one buffer salt is added at at least 0.5 wt. %, advantageously at least 1 wt. %, preferably at least 2 wt. % of salt.

7. Method according to claim 1, characterized in that the bacterial strain is the strain of Leuconostoc mesenteroides B 512.

8. Method according to claim 1, characterized in that fermentation, which comprises the steps of production of the bacterial strain, production of the enzyme and production of dextran, is carried out in the same reactor.

9. Method according to claim 8, characterized in that the reactor is substantially non-sterilized.

10. Solution of substantially native unpurified dextran containing at least 14 wt. %, advantageously at least 18 wt. %, of dextran obtainable by the process according to claim 1.

11. Solution of native dextran according to claim 10, characterized in that it contains at least 2 wt. %, preferably at least 5 wt. %, advantageously at least 8 wt. % of impurities.

12. Solution of native dextran according to claim 1, characterized in that the molecular weight of the dextran is between 100 000 and 500 million g/mol, advantageously 500 000 g/mol and preferably between 1 and 50 million g/mol.

13. Use of the native dextran solution according to claim 11, either as a flocculating agent, coagulating agent, precipitating agent, sedimentation or decanting agent of aqueous suspensions of solid particles, or as a thickener of aqueous media.

14. Use of the native dextran solution according to claim 11, as a flocculant of hydrated alumina resulting from alumina extraction.
